(19) United States
(12) Patent Application Publication
(10) Pub. No.: US 2026/0272929 A1
(43) Pub. Date: Sep. 17, 2026

(54) METHODS FOR TREATING HEPATITIS

(71) Applicant: Aspera Biomedicines, Inc., La Jolla, CA (US)

(72) Inventor: Catriona H. M. Jamieson, La Jolla, CA (US)

(21) Appl. No.: 19/563,945

(22) Filed: Mar. 11, 2026

Related U.S. Application Data
(60) Provisional application No. 63/770,032, filed on Mar. 11, 2025.

(51) Int. Cl. A61K 31/506; A61K 31/365

(52) U.S. Cl. CPC A61K 31/506; A61K 31/365

(57) ABSTRACT

Provided herein are methods for treating HBV and/or HDV infections by administering one or more ADAR inhibitors to a subject in need thereof.

### RELATED APPLICATIONS

This application claims priority of U.S. Provisional Patent Application No. 63/770,032 filed Mar. 11, 2025, the entire content of which is incorporated herein by reference.

### FIELD

Provided herein are methods for inhibiting hepatitis delta (or D) virus replication and/or hepatitis B virus replication.

### BACKGROUND

Hepatitis B is caused by the hepatitis B virus (HBV) and can cause both acute and chronic infections. Hepatitis D is caused by the hepatitis D virus (HDV). Unlike other types of viral hepatitis, HDV cannot cause infection on its own. Also called a satellite virus, it only occurs in individuals who are already infected with the hepatitis B virus (HBV). Hepatitis D affects about 15 million out of the 257 million people suffering from hepatitis B and is rapidly lethal.

Germline-encoded pattern recognition receptors (PRRs) mediate an early innate host immune response against viral pathogens by recognizing interferon (IFN). But HBV is known as a “stealth virus” because little or no IFN production is detected in HBV-infected patients.

The double-stranded RNA-specific adenosine deaminase family of enzymes is encoded by the ADAR family genes. ADAR (adenosine deaminase acting on RNA) proteins enzymatically convert adenosine to inosine, which subsequently disrupts otherwise orthogonal Watson-Crick pairing. Unlike therapeutic use of CRISPR-Cas9, which acts at the gene editing level and is therefore a permanent or inheritable intervention, ADAR enzymes are deaminases that can act on double-stranded RNA (dsRNA), which is a transient molecule. Therefore, ADAR modulation has potential as a transient and tunable, non-heritable, therapeutic intervention.

Several viruses evade the immune system via sequence changes in their dsRNAs thus avoiding recognition by the host immune system. It has been discovered that Adenosine Deaminase Acting on RNA (ADAR) inhibitors can modulate viral immune evasion, which is useful for inhibiting hepatitis delta (or D) virus replication and/or hepatitis B virus replication.

### SUMMARY

Provided herein are methods for inhibition of hepatitis delta virus replication in a subject in need thereof, comprising administration of an Adenosine Deaminase Acting on RNA (ADAR) inhibitor to the subject.

Provided herein are methods for inhibition of immune evasion resulting from A-to-I editing of hepatitis delta viral transcript in a subject in need thereof, comprising administration of an Adenosine Deaminase Acting on RNA (ADAR) inhibitor to the subject.

Provided herein are methods for treating a hepatitis delta virus infection in a subject in need thereof, comprising administration of an Adenosine Deaminase Acting on RNA (ADAR) inhibitor to the subject.

In some embodiments, the subject further suffers from a hepatitis B virus (HBV) infection.

### DETAILED DESCRIPTION

HBV is a DNA virus that infects hepatocytes and replicates through an RNA intermediate. HBV is a small virus that can persist as an episome (covalently closed circular DNA (cccDNA)) in the nucleus of infected cells. Current HBV treatment modalities cannot achieve a complete (sterilizing) cure because the viral DNA integrated into the host genome persists. Seroclearance of HBV surface antigen (HBsAg) is one test that may be used as a surrogate marker for effectiveness of treatment. Seroclearance of HBsAg is considered a functional cure. Only a minority of HBV/HDV patients achieve seroclearance. HDV is an RNA virus that can be found in HBV-infected patients. The majority of patients require a compliance-challenging therapeutic regimen of an indefinite duration of ongoing treatments including, and not limited to, nucleoside or nucleotide analogs, and/or interferon. For example, the World Health Organization (WHO) notes the general recommended treatment for hepatitis D virus infection is at least 48 weeks of pegylated interferon alpha treatment irrespective of the patient's response since the virus tends to give a low rate of response to the treatment. Although such treatment is associated with a lower likelihood of disease progression, it is also associated with significant side effects and should not be given to patients with decompensated cirrhosis, active psychiatric conditions, or autoimmune diseases.

HBV produces transient dsRNA during viral genome transcription and replication. Sequence changes (A-to-G, and U-to-C) characteristic of A-to-I editing occur during virus growth and persistence allowing the virus to evade the immune system.

Described herein are methods that mitigate viral A-to-I editing-based immune evasion. ADAR1 can interact with viral RNAs, such as transient dsRNAs, and deaminate adenosine (A) to generate inosine (I), which can disrupt host immune recognition and perpetuate viral replication. Without being bound by theory, when ADAR1 is inhibited, loss of deaminase activity may prevent or reduce viral A-to-I editing, thereby inhibiting viral replication in the host. The methods described herein may also reduce or prevent persistence of viral DNA integrated into host genomes.

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “inhibition” means a lessening of severity of at least one indicator of a condition or disease (e.g., lessening of viral load, or lessening of viral load to below the limit of detection). The severity of indicators may be determined by subjective or objective measures which are known to those skilled in the art.

The terms “effective amount” and “therapeutically effective amount” refer to an amount of therapeutic compound, or composition, either as a single dose or as part of a series of doses, which is effective to produce a desired therapeutic effect. In general, the therapeutically effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions used in the methods herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two solvents. Lists of suitable salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The term “pharmaceutical composition” refers to a mixture of at least one compound described herein with a pharmaceutically acceptable carrier. The pharmaceutical composition facilitates administration of the compound to a patient or subject. Multiple techniques of administering a compound or composition, exist including, but not limited to, intravenous, oral, aerosol, parenteral, ophthalmic, pulmonary, and topical administration.

The terms “treatment” or “treating” refer to the application of one or more specific procedures used for reducing severity of a disease. A “prophylactic” or “preventive” treatment, which may be a post-exposure prophylactic treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability. When any such inclusion or exclusion occurs, the specification is deemed to contain the group as modified thus fulfilling the written description of all Markush groups used in the appended claims.

References have been made to patents and printed publications throughout this specification, each of which are individually incorporated herein by reference in their entirety.

It is to be understood that the embodiments of this disclosure are illustrative. Accordingly, the present disclosure is not limited to that precisely as shown and described.

### Methods

Provided herein are methods for inhibition of hepatitis delta virus replication in a subject in need thereof, comprising administration of an Adenosine Deaminase Acting on RNA (ADAR) inhibitor to the subject.

Provided herein are methods for inhibition of immune evasion resulting from A-to-I editing of hepatitis delta viral transcript in a subject in need thereof, comprising administration of an Adenosine Deaminase Acting on RNA (ADAR) inhibitor to the subject.

In some embodiments, the methods herein reduce A-to-I editing of hepatitis delta viral transcript in a subject by about 10% to about 99.9%. In some embodiments, the methods herein reduce A-to-I editing of hepatitis delta viral transcript in a subject by about 20% to about 99%, by about 50% to about 95%, or by about 70% to about 95%.

In some embodiments, after administration of an ADAR inhibitor, viral replication of hepatitis delta virus is reduced by about 10% to about 99.9%, by about 20% to about 99%, by about 50% to about 95%, or by about 70% to about 95%, or HDV DNA is not measurable/detectable. In some embodiments, after administration of an ADAR inhibitor, hepatic inflammation is reduced, as measured by serum aminotransferases alanine transaminase (ALT) and/or aspartate transaminase (AST) levels, by about 10% to about 99.9%, by about 20% to about 99%, by about 50% to about 95%, or by about 70% to about 95%, indicating reduction in HDV viral replication. In some embodiments, an ADAR inhibitor is administered to hepatitis B e antigen (HBeAg)-positive subjects, and after the administration, a reduction of anti-HBe (or HBeAb, Hepatitis B e-Antibody) is detected, wherein the reduction is about 10% to about 99.9%, about 20% to about 99%, about 50% to about 95%, or about 70% to about 95%, compared to levels of anti-HBe prior to treatment with an ADAR inhibitor, thereby indicating reduced HDV replication. Hepatitis B e antigen (HBeAg) is a secretory protein processed from the pre-core protein. It may be used as a marker of HBV replication and infectivity. In some embodiments of the methods herein, its presence may be associated with the presence of HBV DNA.

Provided herein are methods for treating a hepatitis delta virus infection in a subject in need thereof, comprising administration of an Adenosine Deaminase Acting on RNA (ADAR) inhibitor to the subject.

In some embodiments of the methods herein, the subject comprises an acute hepatitis B virus (HBV) infection. In some embodiments of the methods herein, the subject comprises a chronic hepatitis B virus (HBV) infection.

Provided herein are methods for inhibition of hepatitis B virus replication in a subject in need thereof, comprising administration of an Adenosine Deaminase Acting on RNA (ADAR) inhibitor to the subject, wherein the inhibition includes inhibition of immune evasion resulting from A-to-I editing of hepatitis B viral transcript.

Provided herein are methods for inhibition of immune evasion resulting from A-to-I editing of hepatitis B viral transcript in a subject in need thereof, comprising administration of an Adenosine Deaminase Acting on RNA (ADAR) inhibitor to the subject.

In some embodiments, after administration of an ADAR inhibitor, viral replication of hepatitis B virus is reduced by about 10% to about 99.9%, by about 20% to about 99%, by about 50% to about 95%, or by about 70% to about 95%. In some embodiments, after administration of an ADAR inhibitor, hepatic inflammation is reduced, as measured by serum aminotransferases alanine transaminase (ALT) and/or aspartate transaminase (AST) levels, by about 10% to about 99.9%, by about 20% to about 99%, by about 50% to about 95%, or by about 70% to about 95%, indicating reduction in HBV viral replication. In some embodiments, an ADAR inhibitor is administered to hepatitis B e antigen (HBeAg)-positive subjects, and after the administration, a reduction of anti-HBe (or HBeAb, Hepatitis B e-Antibody) is detected, wherein the reduction is about 10% to about 99.9%, about 20% to about 99%, about 50% to about 95%, or about 70% to about 95%, compared to levels of anti-HBe prior to treatment with an ADAR inhibitor, thereby indicating reduced HBV replication.

Provided herein are methods for treating a hepatitis B virus infection in a subject in need thereof, comprising administration of an Adenosine Deaminase Acting on RNA (ADAR) inhibitor to the subject.

In some embodiments of the methods herein, the subject comprises an acute hepatitis B virus (HBV) infection. In some embodiments of the methods herein, the subject comprises a chronic hepatitis B virus (HBV) infection.

In some embodiments, the subject further comprises a hepatitis delta virus infection.

In some embodiments, after administration of an ADAR inhibitor, viral replication of HBV and HDV is reduced by about 10% to about 99.9%, by about 20% to about 99%, by about 50% to about 95%, or by about 70% to about 95%. In some embodiments, after administration of an ADAR inhibitor, hepatic inflammation is reduced, as measured by serum aminotransferases alanine transaminase (ALT) and/or aspartate transaminase (AST) levels, by about 10% to about 99.9%, by about 20% to about 99%, by about 50% to about 95%, or by about 70% to about 95%, indicating reduction in HBV and HDV viral replication. In some embodiments, an ADAR inhibitor is administered to hepatitis B e antigen (HBeAg)-positive subjects, and after the administration, a reduction of anti-HBe (or HBeAb, Hepatitis B e-Antibody) is detected, wherein the reduction is about 10% to about 99.9%, about 20% to about 99%, about 50% to about 95%, or about 70% to about 95%, compared to levels of anti-HBe prior to treatment with an ADAR inhibitor, thereby indicating reduced HBV and HDV replication.

In some embodiments, the subject has previously undergone treatment for HDV and/or HBV. In some embodiments, the subject is a treatment naïve subject.

Provided herein are methods for treatment of hepatitis B virus (HBV) infection in a subject in need thereof, the method comprising administering a therapeutically effective amount of an Adenosine deaminase acting on RNA (ADAR) inhibitor to the subject.

Provided herein are methods for treatment of acute hepatitis B virus (HBV) infection in a subject in need thereof, the method comprising administering a therapeutically effective amount of an Adenosine deaminase acting on RNA (ADAR) inhibitor to the subject.

Provided herein are methods for treatment of chronic hepatitis B virus (HBV) infection in a subject in need thereof, the method comprising administering a therapeutically effective amount of an Adenosine deaminase acting on RNA (ADAR) inhibitor to the subject.

Provided herein are methods for treatment of liver cirrhosis from hepatitis B virus (HBV) infection in a subject in need thereof, the method comprising administering a therapeutically effective amount of an Adenosine deaminase acting on RNA (ADAR) inhibitor to the subject.

Provided herein are methods for treatment of liver cancer (e.g., hepatocellular carcinoma) from hepatitis B virus (HBV) infection in a subject in need thereof, the method comprising administering a therapeutically effective amount of an Adenosine deaminase acting on RNA (ADAR) inhibitor to the subject.

In some embodiments of the methods herein, the subject further comprises a hepatitis delta virus (HDV) infection.

In some embodiments of the methods herein, the subject is suffering from or diagnosed with concurrent HDV infection with fulminant acute hepatitis B virus (HBV) infection (acute coinfection), chronic HBV infection (chronic coinfection), or acute exacerbation of known chronic HBV infection (HDV superinfection).

Provided herein are methods for reducing expression of small-HDAg or large-HDAg from HDV infection in a subject in need thereof, the method comprising administering a therapeutically effective amount of an Adenosine deaminase acting on RNA (ADAR) inhibitor to the subject.

Provided herein are methods for inhibiting HDV virion assembly in a subject in need thereof, the method comprising administering a therapeutically effective amount of an Adenosine deaminase acting on RNA (ADAR) inhibitor to the subject.

Provided herein are methods for the treatment of chronic hepatitis delta virus (HDV) infection in a plasma (or serum) HDV-RNA positive adult subject with compensated liver disease, the method comprising administering a therapeutically effective amount of an Adenosine deaminase acting on RNA (ADAR) inhibitor (e.g., Compound A) to the subject.

In some embodiments, administration of an ADAR inhibitor inhibits immune evasion resulting from A-to-I editing of hepatitis B viral transcript. In some embodiments, administration of an ADAR inhibitor inhibits immune evasion resulting from A-to-I editing of hepatitis delta viral transcript. In some embodiments, the method inhibits immune evasion by the HBV virus and the HDV virus by inhibiting Adenosine-to-Inosine (A-to-I) editing in the viral RNAs. In some embodiments, administration of an ADAR inhibitor improves biomarkers of liver fibrosis and/or cirrhosis (e.g., ALT, AST, ratio of AST/ALT, alkaline phosphatase (ALP)).

In some embodiments, a reduction in A-to-I editing of a HBV and/or HDV viral transcript may correlate with a reduction in HBV and/or HDV viral load. In some embodiments, HBV and/or HDV is measured/detected by a blood test such as Anti-HBc IgM or anti-HBc IgG (anti-hepatitis B core IgM or IgG) test, where a positive/reactive test indicates an infection. In some embodiments, HBV and/or HDV viral load is measured by a blood test such as an HBeAg (Hepatitis B e-Antigen) test, where a positive test indicates high levels of virus in blood. In some embodiments, HBV and/or HDV viral load is measured by a blood test such as viral DNA quantification (“viral load”). The viral load is usually measured in “international units per milliliter” (IU/mL), but may also be measured in “copies per milliliter” (cp/mL). There are approximately 5 copies in one international unit. In some embodiments, HBV and/or HDV viral load is measured by a blood test such as a HBsAg Quantitative (quantitative hepatitis B surface antigen/qHBsAg) test, where the actual amount of hepatitis B surface antigen in the blood is measured.

In some embodiments, the methods described herein provide a functional cure of HBV and/or HDV infection. Functional cure of HBV refers to undetectable hepatitis B surface antigen (HBsAg) and undetectable HBV virus DNA after 6 months off therapy (see Lim, S. G., et al., Nat Rev Gastroenterol Hepatol 2023, 20, 238-253). In some embodiments, functional cure includes <100 IU/mL of viral load.

In some embodiments of the methods herein, an ADAR inhibitor may be administered in combination with an additional therapeutic such as an HBV polymerase inhibitor, a capsid inhibitor, a pegylated interferon, an immunotherapeutic, or any combination thereof.

In some embodiments of the methods described herein, the ADAR inhibitor is selected from

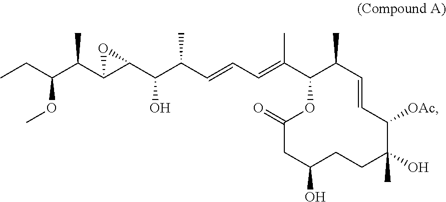 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]

ruxolitinib (e.g., ruxolitinib phosphate), N-tert-butyl-3-[(5-methyl-2-{[4-(2-pyrrolidin-1-ylethoxy)phenyl]amino}pyrimidin-4-yl)amino]benzenesulfonamide (e.g., fedratinib) (fedratinib is a kinase inhibitor with the chemical name N-tert-butyl-3-[(5-methyl-2-{[4-(2-pyrrolidin-1-ylethoxy)phenyl]amino}pyrimidin-4-yl)amino]benzenesulfonamide dihydrochloride monohydrate), a pladienolide (e.g., pladienolide B), dasatinib, raltegravir (e.g., raltegrabir potassium), dolutegravir (e.g., dolutregravir sodium), AVA-ADR-001, ZYS-1, cladribine, 8-azaadenosine, pentostatin, EHNA (e.g., as a hydrochloride salt), AMPD2 inhibitor 1, hibifolin, coformycin, FR221647, FR234938, AMPD2 inhibitor 2, or a pharmaceutically acceptable salt thereof.

In some embodiments of the methods herein, the ADAR inhibitor is Compound A.

In some embodiments of the methods herein, the ADAR inhibitor is fedratinib or N-tert-butyl-3-[(5-methyl-2-{[4-(2-pyrrolidin-1-ylethoxy)phenyl]amino}pyrimidin-4-yl)amino]benzenesulfonamide or a pharmaceutically acceptable salt thereof. Fedratinib is an oral kinase inhibitor with activity against wild type and mutationally activated Janus Associated Kinase 2 (JAK2) and FMS-like tyrosine kinase 3 (FLT3). Fedratinib is a JAK2-selective inhibitor with higher inhibitory activity for JAK2 over family members JAK1, JAK3 and TYK2.

In some embodiments, the methods described herein further comprise administration of a second ADAR inhibitor to the subject. In some embodiments, the methods described herein comprise administration of Compound A and fedratinib to the subject.

In some embodiments, the methods described herein comprise administration of about 25 to about 400 mg of Compound A (e.g., once daily), about 25 to about 400 mg of fedratinib (e.g., once daily), or about 25 to about 400 mg of fedratinib (e.g., once daily) and about 25 to about 400 mg of Compound A (e.g., each, independently, once daily).

In some embodiments, the methods described herein further comprise administration of one or more additional HDV antiviral agents to the subject, e.g., bulevirtide (e.g., bulevirtide acetate), lonafamib, myrcludex B, ezetimibe, REP 2055, REP 2139, REP 2165, GI-18000, ALN-HDV, pegylated interferon alpha, pegylated interferon lambda, or a pharmaceutically acceptable salt thereof, or a combination thereof. Thus, in some embodiments, the methods herein may include administration of a combination of a first HDV therapeutic agent selected from an ADAR inhibitor described herein and a second HDV therapeutic agent selected from bulevirtide (e.g., bulevirtide acetate), lonafarnib, myrcludex B, ezetimibe, REP 2139, REP 2165, GI-18000, ALN-HDV, pegylated interferon alpha, pegylated interferon lambda, or a combination thereof. In some embodiments, the first and second HDV therapeutic agent are simultaneously administered or sequentially administered.

In some embodiments, the methods described herein further comprise administration of one or more additional HBV antiviral agents to the subject, e.g., pegylated interferon alpha, pegylated interferon lambda, lamivudine, adefovir dipivoxil, entecavir, telbivudine, tenofovir, tenofovir alfenamide, cledvudine, elebsiran, xalnisiran, imdurisan, BW-20507, ALG-125755, BB-103, JNJ-3989, HT-101, bulevirtide, capsid inhibitors (e.g., ZM-H1505R, EDP-514, ALG-000184, ABI-H4334, CS12088), REP 2139, bepirovirsen, AHB-137, EBT107, PBGENE-HBV, TUNE-401, or a combination thereof.

In some embodiments of the methods herein, HBV infection is acute HBV infection. In some embodiments, HBV infection is chronic hepatitis B (CHB), and may be associated with liver disease, including liver inflammation, fibrosis, cirrhosis and/or liver cancer.

In some embodiments of the methods herein, the subject is human. In some embodiments, the human subject is an adult. In some embodiments, the human subject is a pre-natal subject. In some embodiments, the human subject is a neo-natal subject. In some embodiments, the subject suffers from HBV infection. In some embodiments, the subject further suffers from HDV infection. In some embodiments, the subject suffers from a reactivated HBV infection (e.g., a reactivation after a period of functional cure). In some embodiments, the subject suffers from HDV infection and HBV infection.

In some embodiments of the methods described herein, the administration is oral administration. In some embodiments of the methods described herein, the administration is parenteral administration.

In some embodiments of the methods described herein, the ADAR inhibitor is administered as a lipid nanoparticle composition. In some embodiments, the lipid nanoparticle composition comprises a cationic lipid, a polyethyleneglycol (PEG)-modified lipid, a sterol, and a non-cationic lipid. In some embodiments, the cationic lipid is an ionizable cationic lipid, the non-cationic lipid is a neutral lipid, and the sterol is a cholesterol. In some embodiments, the lipid nanoparticle composition comprises an ionizable cationic lipid, a polyethyleneglycol (PEG)-modified lipid, a cholesterol, and a neutral lipid.

The methods described herein may occur in vivo or in vitro, including within a subject, such as a human subject. In some embodiments, the methods are applied to a cell in vitro. In some embodiments, the methods are applied to a cell in vivo, e.g., applied to a subject, e.g., in a mammalian subject, e.g., a human subject.

Actual dosage levels of the active ingredient ADAR inhibitor (e.g., Compound A) may be independently varied to obtain amounts of the active ingredients effective to achieve the desired therapeutic response for a particular patient, composition, and/or mode of administration, without being toxic to the patient.

In particular, the selected dosage levels will depend upon a variety of factors including the activity of the particular compound employed, the time of administration, the rate of excretion of the compound, the duration of the treatment, other drugs, compounds or materials used by the patient, the age, sex, weight, condition, general health, and prior medical history of the patient being treated, and like factors well-known in the medical arts. A medical doctor, e.g., physician or veterinarian, having ordinary skill in the art may readily determine and prescribe the effective amount of the pharmaceutical composition(s) required. For example, the physician or veterinarian could start doses of the compounds employed in the pharmaceutical composition(s) at levels lower than that required in order to achieve the desired therapeutic effect and gradually increase the dosage until the desired effect is achieved.

In some embodiments of the methods described herein include administration of compositions comprising one or more ADAR inhibitor(s). In some embodiments of the methods herein, the compositions administered may be pharmaceutical compositions further comprising a pharmaceutically acceptable carrier. In some embodiments of the methods herein, one or more ADAR inhibitors selected from ADAR1 inhibitors may be administered. In some embodiments of the methods herein, the ADAR inhibitor administered is an inhibitor of ADAR1 p150 isoform present in cytoplasm. In some embodiments of the methods herein, the ADAR inhibitor administered is selected from a pladienolide (including pladienolides A, B, C, D, E, F, and G), Compound A, Compound B, fedratinib, ruxolitinib, AVA-ADR-001, ZYS-1, cladribine, 8-azaadenosine, pentostatin, EHNA hydrochloride, AMPD2 inhibitor 1, hibifolin, coformycin, FR221647, FR234938, or AMPD2 inhibitor 2, or a pharmaceutically acceptable salt thereof. In some embodiments, the ADAR inhibitor is selected from one or more of dasatinib, raltegravir, dolutegravir, pladienolide B, Compound A, Compound B, ruxolitinib, or fedratinib, or a pharmaceutically acceptable salt thereof. In some embodiments of the methods herein, the ADAR inhibitor administered is selected from pladienolides (including pladienolides A, B, C, D, E, F, and G), Compound A, Compound B, or AVA-ADR-001, or a pharmaceutically acceptable salt thereof. In some embodiments of the methods herein, the ADAR inhibitor administered is Compound A.

The ADAR inhibitors used in the methods herein are commercially available or are prepared according to previously described synthetic procedures and incorporated into compositions.

For example, Compound A has been described, including in WO 2021/026273 A1 and U.S. Pat. No. 10,675,267 B2, which are incorporated herein by reference. Compound A has also been synthesized as described by Chan et al. (Cell Reports Physical Science, 2020, 1, 12, 100277); Compound B is similarly prepared.

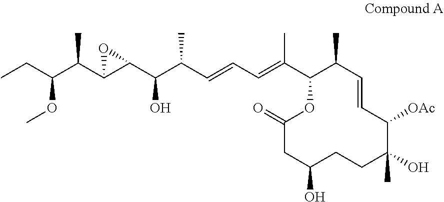 [SMILES: CC[C@H](OC)[C@@H](C)[C@H]1O[C@@H]1[C@H](O)[C@H](C)/C=C/C=C(\C)[C@H]1OC(=O)C[C@H](O)CC[C@@](C)(O)[C@@H](OC(C)=O)/C=C/[C@@H]1C]

Pladienolide B has been synthesized previously, at least, as described, for example, by Rhoades et al. (Journal of the American Chemical Society 2021 143 (13), 4915-4920 DOI: 10.1021/jacs.1c01135). Pladienolides A, C, D, E, F, and G are similarly prepared.

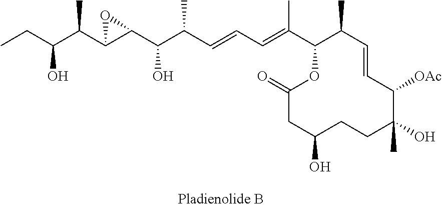 [SMILES: CC[C@H](O)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]

The compounds used in the methods described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as described herein are modified using appropriate reagents and conditions, for the introduction of the various moieties found in the formula as provided herein.

The compounds used in the methods herein are synthesized using any suitable procedures starting from compounds that are available from commercial sources or are prepared using procedures described herein.

In some embodiments, the ADAR inhibitor used in the methods described herein includes compounds that may be isotopically-labeled compounds (e.g., isotopically-labeled ADAR inhibitors), which are identical to those recited in the formulae herein, but for the fact that one or more atoms are replaced by an atom having an atomic mass or mass number abundance different from the atomic mass or mass number abundance usually found in nature. Examples of isotopes suitable for inclusion in the compounds of compositions herein include, without limitation, hydrogen, carbon, nitrogen, oxygen, and fluorine, such as, but not limited to ²H, ³H, ¹³C, ¹⁴C, ¹⁵N, ¹⁷O, ¹⁸O, and ¹⁸F, respectively. Other isotopes are contemplated herein, for example those of P, Cl, I, etc. Substitution with heavier isotopes such as deuterium, i.e., ²H, can afford certain therapeutic advantages resulting from greater metabolic stability, for example, increased in vivo half-life or reduced dosage requirements and hence may be preferred in some circumstances. A compound in the compositions herein may incorporate positron-emitting isotopes for medical imaging and positron-emitting tomography (PET) studies for determining the distribution of receptors. Suitable positron-emitting isotopes that can be incorporated in the compounds herein are ¹¹C, ¹³N, ¹⁵O, and ¹⁸F. Compounds herein that are isotopically-labeled can generally be prepared by conventional techniques known to those skilled in the art or by processes analogous to those described herein using appropriate isotopically-labeled reagents or starting materials in place of non-isotopically-labeled reagents or starting materials. Thus, in some embodiments, provided herein are compositions comprising compounds of the formulae herein, wherein at least one atom is replaced by its corresponding atom having an atomic mass or mass number abundance different from the atomic mass or mass number abundance usually found in nature. For example, the compositions comprise compounds having one or more H (e.g., ¹H) substituted, independently, with ²H or ³H. In some embodiments, one or more C is substituted, independently, with ¹³C or ¹⁴C. In some embodiments, one or more N is substituted with ¹⁵N. In some embodiments, one or more 0 is substituted, independently, with ¹⁷O or ¹⁸O. In some embodiments, one or more F is substituted, independently, with ¹⁸F.

Routes of administration of the ADAR inhibitors herein may include, without limitation, oral, nasal, rectal, intravaginal, parenteral, buccal, sublingual, or topical administration. In some embodiments, the oral or nasal route of administration is an oral inhalational or nasal inhalational route of administration. The compounds for use in the methods herein may be formulated for administration by any suitable route to achieve the particular method being applied.

In some embodiments an ADAR inhibitor used in the methods herein is incorporated in lipid nanoparticles (LNPs). In some embodiments, the LNPs comprise a lipid core matrix that can solubilize lipophilic molecules. The lipid core is stabilized by surfactants (emulsifiers). The emulsifier chosen depends on the route of administration. In some embodiments, the core lipids may be fatty acids, acylglycerols, waxes, or combinations thereof. Biological membrane lipids such as phospholipids, sphingomyelins, bile salts (sodium taurocholate), and sterols (cholesterol) may be used as stabilizers. In some embodiments, LNPs may comprise an ionizable cationic lipid (whose positive charge binds to a negative charge on the compound), a PEGylated lipid (for stability), a phospholipid (for structure), and cholesterol (for structure). In some embodiments, LNPs comprise a cationic lipid, a PEG-modified lipid, a sterol, and a non-cationic lipid. In some embodiments, the cationic lipid is an ionizable cationic lipid, the non-cationic lipid is a neutral lipid, and the sterol is a cholesterol. In some embodiments, LNPs are used for intravenous delivery of an ADAR inhibitor.

In some embodiments of the methods herein, solid lipid nanoparticles (SLNs) may be used. SLNs may be used for oral administration of an ADAR inhibitor and can provide intestinal delivery of the ADAR inhibitor.

In some embodiments of the methods herein, an ADAR inhibitor composition is used for oral administration. In some embodiments, an ADAR inhibitor is formulated as a tablet, a capsule, a lozenge (e.g., a troche), a syrup, a dispersible powder to be mixed with food, or any other suitable oral composition.

In some embodiments of the methods herein, provided herein are packaged compositions, or packaged pharmaceutical compositions, comprising a container holding a therapeutically effective amount of an ADAR inhibitor described herein, such as, but not limited to, fedratinib, ruxolitinib, Compound A, or pladienolide B, or a pharmaceutically acceptable salt thereof, or the like, and instructions for using the compositions in accordance with one or more of the methods provided herein.

The ADAR compositions, and associated materials used in the methods herein can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g. Irgacure 184, 2959), preferably water-soluble initiators (e.g. Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g. by the addition of specific product information leaflets) into suitable containers (boxes, etc.).

According to further embodiments, the ADAR compositions used in the methods herein can also be provided in kit form combined with other components necessary for administration of the material to the patient. For example, disclosed kits, such as for use in the treatment of HBV and/or HDV, can further comprise, for example, administration materials.

The kits are designed in various forms based on the specific deficiencies they are designed to treat.

The ADAR compositions used in the methods herein may be prepared and placed in a container for storage at ambient or elevated temperature. When the ADAR composition is stored in a polyolefin plastic container as compared to a polyvinyl chloride plastic container, discoloration of the ADAR composition, or sorption of the ADAR inhibitor with the surface of the container, may be reduced, whether dissolved or suspended in a liquid composition (e.g., an aqueous or organic liquid solution), or as a solid. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce exposure of the container's contents to infrared light, or also include a second component with such a capacity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper, cardboard, paperboard, metallic film, or foil, or a combination thereof, container to further reduce exposure of the container's contents to UV, visible, or infrared light. The ADAR compositions provided herein may need storage lasting up to, or longer than, three months; in some cases, up to, or longer than one year. The containers may be in any form suitable to contain the contents, for example, a bag, a bottle, or a box.

The following examples further illustrate aspects of the present disclosure. However, they are in no way a limitation of the teachings or disclosure as described herein.

### Examples

### Example 1: Assay for Measuring Viral Load

Kinetics or fitness of hematopoietic stem cells in a subject challenged with HBV/HDV. Effects of Compound A administration to the subject on hematopoietic stem cell fitness or kinetics is measured by confocal fluorescent microscopic tracking of HSPC cell cycle kinetics with a dual fluorescence lentiviral FUCCI2BL cell cycle indicator (Pineda . . . Jamieson. Scientific Reports 2016), quantification of ADAR1 self-renewal gene activity with a lentiviral ADAR1 GFP reporter, and cell fate decisions in response to HBV/HDV challenge for up to a 4 to 6 week period or longer (Blood 144 (2024) 4068-4069).

### Example 2: HBV Treatment

A subject is administered a pharmaceutical composition comprising about 25 to about 400 mg of an ADAR inhibitor once daily, which results in reduction of HBV viral load as determined by binary detection (e.g., positive or negative result) or quantification of one or more of a hepatitis B viral DNA (e.g., by PCR), an anti-HBc or HBcAb (heptatitis B core antibody), an anti-HBs or HBsAb (hepatitis B surface antibody), an HBeAg (hepatitis B e antigen), an IgM anti-HBc, an IgG anti-HBc, or an HBsAg (hepatitis B surface antigen) (e.g., by ELISA) in the subject's blood. Alternatively, quantification of assays described in Example 1 are utilized to characterize impact of ADAR inhibition therapy on reduction of HBV or HDV symptoms or viral load.

### Example 3: HDV Treatment

A subject is administered a pharmaceutical composition comprising about 25 to about 400 mg of an ADAR inhibitor once daily, which results in reduction of HDV viral load as determined by binary detection (e.g., positive or negative result) quantification of a hepatitis D viral RNA (e.g., by PCR), an IgM anti-HDV-Ab, or an IgG anti-HDV-AB in the subject's blood. Alternatively, quantification of assays described in Example 1 are utilized to characterize impact of ADAR inhibition therapy on reduction of HBV or HDV symptoms or viral load.

### Example 4: Assay for Measuring Viral Load

Kinetics or fitness of hematopoietic stem cells in a subject challenged with HBV/HDV. Effects of fedratinib administration to the subject on hematopoietic stem cell fitness or kinetics is measured by confocal fluorescent microscopic tracking of HSPC cell cycle kinetics with a dual fluorescence lentiviral FUCCI2BL cell cycle indicator (Pineda . . . Jamieson. Scientific Reports 2016), quantification of ADAR1 self-renewal gene activity with a lentiviral ADAR1 GFP reporter, and cell fate decisions in response to HBV/HDV challenge for up to a 4 to 6 week period or longer (Blood 144 (2024) 4068-4069).

### Example 5: HBV Treatment

A subject is administered a pharmaceutical composition comprising about 25 to about 400 mg of fedratinib or about 25 to about 400 mg of Compound A, or both, once daily, which results in reduction of HBV viral load as determined by binary detection (e.g., positive or negative result) or quantification of one or more of a hepatitis B viral DNA (e.g., by PCR), an anti-HBc or HBcAb (heptatitis B core antibody), an anti-HBs or HBsAb (hepatitis B surface antibody), an HBeAg (hepatitis B e antigen), an IgM anti-HBc, an IgG anti-HBc, or an HBsAg (hepatitis B surface antigen) (e.g., by ELISA) in the subject's blood. Alternatively, quantification of assays described in Example 1 or 4 are utilized to characterize impact of the administered therapy on reduction of HBV or HDV symptoms or viral load.

### Example 6: HDV Treatment

A subject is administered a pharmaceutical composition comprising about 25 to about 400 mg of fedratinib or about 25 to about 400 mg of Compound A, or both, once daily, which results in reduction of HDV viral load as determined by binary detection (e.g., positive or negative result) quantification of a hepatitis D viral RNA (e.g., by PCR), an IgM anti-HDV-Ab, or an IgG anti-HDV-AB in the subject's blood. Alternatively, quantification of assays described in Example 1 and 4 are utilized to characterize impact of the administered therapy on reduction of HBV or HDV symptoms or viral load.

1. (canceled)

2. (canceled)

3. A method of treating a hepatitis delta virus infection in a subject in need thereof, comprising administration of an Adenosine Deaminase Acting on RNA (ADAR) inhibitor to the subject.

4. The method of claim 3, wherein the subject comprises an acute hepatitis B virus (HBV) infection.

5. The method of claim 3, wherein the subject comprises a chronic hepatitis B virus (HBV) infection.

6-11. (canceled)

12. The method of claim 3, wherein the ADAR inhibitor is selected from ruxolitinib, N-tert-butyl-3-[(5-methyl-2-{[4-(2-pyrrolidin-1-ylethoxy)phenyl]amino}pyrimidin-4-yl)amino]benzenesulfonamide, a pladienolide, dasatinib, raltegravir, dolutegravir AVA-ADR-001, ZYS-1, cladribine, 8-azaadenosine, pentostatin, EHNA, AMPD2 inhibitor 1, hibifolin, coformycin, FR221647, FR234938, or AMPD2 inhibitor 2, or a pharmaceutically acceptable salt thereof.

13. The method of claim 3, wherein the ADAR inhibitor is

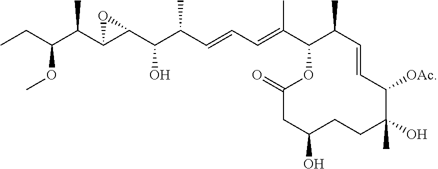 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]

14. The method of claim 3, wherein the ADAR inhibitor is N-tert-butyl-3-[(5-methyl-2-{[4-(2-pyrrolidin-1-ylethoxy)phenyl]amino}pyrimidin-4-yl)amino]benzenesulfonamide or a pharmaceutically acceptable salt thereof.

15. The method of claim 3, further comprising administration of a second ADAR inhibitor to the subject, wherein the second ADAR inhibitor is selected from ruxolitinib, N-tert-butyl-3-[(5-methyl-2-{[4-(2-pyrrolidin-1-ylethoxy)phenyl]amino}pyrimidin-4-yl)amino]benzenesulfonamide, a pladienolide, dasatinib, raltegravir, dolutegravir, AVA-ADR-001, ZYS-1, cladribine, 8-azaadenosine, pentostatin, EHNA, AMPD2 inhibitor 1, hibifolin, coformycin, FR221647, FR234938, or AMPD2 inhibitor 2, or a pharmaceutically acceptable salt thereof.

16. The method of claim 3, wherein the administration is oral administration.

17. The method of claim 3, wherein the ADAR inhibitor is administered in a form of a lipid nanoparticle composition.

18. The method of claim 17, wherein the lipid nanoparticle composition comprises a cationic lipid, a polyethyleneglycol (PEG)-modified lipid, a sterol, and a non-cationic lipid.

19. The method of claim 17, wherein the lipid nanoparticle composition comprises an ionizable cationic lipid, a polyethyleneglycol (PEG)-modified lipid, a cholesterol, and a neutral lipid.

20. The method of claim 3, wherein the ADAR inhibitor is 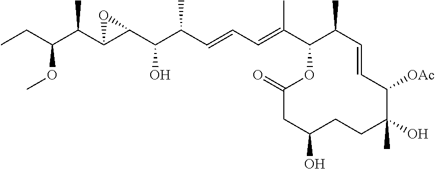 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]
and further comprising administration of a second ADAR inhibitor to the subject, wherein the second ADAR inhibitor is N-tert-butyl-3-[(5-methyl-2-{[4-(2-pyrrolidin-1-ylethoxy)phenyl]amino}pyrimidin-4-yl)amino]benzenesulfonamide or a pharmaceutically acceptable salt thereof.

21. The method of claim 3, comprising administration of 25 to 400 mg of the ADAR inhibitor once daily to the subject, wherein the ADAR inhibitor is 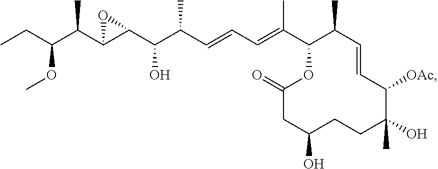 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]
and
further comprising administration of 25 to 400 mg of a second ADAR inhibitor once daily to the subject, wherein the second ADAR inhibitor is N-tert-butyl-3-[(5-methyl-2-{[4-(2-pyrrolidin-1-ylethoxy)phenyl]amino}pyrimidin-4-yl)amino]benzenesulfonamide or a pharmaceutically acceptable salt thereof.
